SEQUENCE LISTING (11 sequences)

SEQ ID NO: 1 - protein, 372 aa, Homo sapiens
MEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 2 - protein, 372 aa, Homo sapiens
AEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 3 - protein, 372 aa, Homo sapiens
MEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 4 - protein, 372 aa, Homo sapiens
AEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 5 - protein, 372 aa, Homo sapiens; identical to SEQ ID NO: 4

SEQ ID NO: 6 - protein, 372 aa, Homo sapiens
AEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVGFFLVLDVVYLVYESKHLHEGAKSETAEELKKVAQELEEKLNMLNNNYKILQADQEL

SEQ ID NO: 7 - protein, 371 aa, Homo sapiens
MAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNKILQADQEL

SEQ ID NO: 8 - protein, 370 aa, Homo Sapiens
AEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNKILQADQEL

SEQ ID NO: 9 - protein, 362 aa, Papio hamadryas
MEEATARVQQNHPGGTDTGDPQESNVSLEDYLNYFQKNVSPQEMLLLLSDHKAWERVVATAELPRDEADELYKALNKLIRHMVMKDKNWLEEVQQHRKRFLEEFPRLERELQDKIRRLCDLAGQVQKVHKGATIANAFSSTLGVASGVLTFLGLGLAPFTAGSSLVLLEPVTGLGIAAALTGITSGSVEYAKKRWAQAEAHELVNKSLDTVEEMNEFLYHNIPNFISLRVNLVKFTEDTGKAIRAIRQARANPHSVSHVPASLHRVTEPVSATSVEERARVVEMERVAESRTTEVIRGAKIVDKVFEGALFVLDVVGLVCQLKHLHEGAKSKTAEELKKVAQELEKKLNILNKKYETLRQEP

SEQ ID NO: 10 - protein, 362 aa, Papio hamadryas
AEEATARVQQNHPGGTDTGDPQESNVSLEDYLNYFQKNVSPQEMLLLLSDHKAWERVVATAELPRDEADELYKALNKLIRHMVMKDKNWLEEVQQHRKRFLEEFPRLERELQDKIRRLCDLAGQVQKVHKGATIANAFSSTLGVASGVLTFLGLGLAPFTAGSSLVLLEPVTGLGIAAALTGITSGSVEYAKKRWAQAEAHELVNKSLDTVEEMNEFLYHNIPNFISLRVNLVKFTEDTGKAIRAIRQARANPHSVSHVPASLHRVTEPVSATSVEERARVVEMERVAESRTTEVIRGAKIVDKVFEGALFVLDVVGLVCQLKHLHEGAKSKTAEELKKVAQELEKKLNILNKKYETLRQEP

SEQ ID NO: 11 - protein, 337 aa, Homo sapiens
MNPESSIFIEDYLKYFQDQVSRENLLQLLTDDEAWNGFVAAAELPRDEADELRKALNKLASHMVMKDKNRHDKDQQHRQWFLKEFPRLKRELEDHIRKLRALAEEVEQVHRGTTIANVVSNSVGTTSGILTLLGLGLAPFTEGISFVLLDTGMGLGAAAAVAGITCSVVELVNKLRARAQARNLDQSGTNVAKVMKEFVGGNTPNVLTLVDNWYQVTQGIGRNIRAIRRARANPQLGAYAPPPHIIGRISAEGGEQVERVVEGPAQAMSRGTMIVGAATGGILLLLDVVSLAYESKHLLEGAKSESAEELKKRAQELEGKLNFLTKIHEMLQPGQDQ

(19) United States
(12) Patent Application Publication
(10) Pub. No.: US 2015/0307591 A1
(43) Pub. Date: Oct. 29, 2015

(54) ON-COLUMN REFOLDING AND PURIFYING OF LIPOPROTEINS

(71) Applicant: MERCK SHARP & DOHME CORP., Rahway, NJ (US)

(72) Inventors: Rebecca A. Chmielowski, Scotch Plains, NJ (US); Collette Cutler, Bloomingdale, NJ (US); Hong Li, Basking Ridge, NJ (US); Thomas O. Linden, Watchung, NJ (US)

(73) Assignee: MERCK SHARP & DOHME CORP., Rahway, NJ (US)

(21) Appl. No.: 14/653,620

(22) PCT Filed: Dec. 19, 2013
(86) PCT No.: PCT/US13/76324

§ 371 Date: Jun. 18, 2015

Related U.S. Application Data
(60) Provisional application No. 61/740,952, filed on Dec. 21, 2012.

(51) Int. Cl. C07K 14/775; C07K 1/20

(52) U.S. Cl. CPC C07K 14/775; C07K 1/20

(57) ABSTRACT

Provided are methods of refolding and purifying lipoproteins on a chromatography column. In particular methods of refolding ApoL1 polypeptides using a hydrophobic interaction column (HIC). The present invention provides methods for the purification of an active form of a human lipoprotein, such as ApoL1. More particularly, the present invention relates to a method for renaturing an inclusion body of proteins expressed in a large quantity in E. coli into an active form using a hydrophobic interaction column and removal of impurities, e.g., endotoxins.

### FIELD OF THE INVENTION

The present invention provides methods for the purification of an active form of a human lipoprotein, such as ApoL1. More particularly, the present invention relates to a method for renaturing an inclusion body of proteins expressed in a large quantity in E. coli into an active form using a hydrophobic interaction column and removal of impurities, e.g., endotoxins.

### BACKGROUND OF THE INVENTION

Manufacturing of recombinant therapeutic proteins pose many purification challenges including removal of process and product related impurities, such as endotoxin, host cell proteins, and protein fragments. These challenges are amplified with production of apolipoproteins since these hydrophobic proteins tend to have a high binding affinity for impurities and will co-purify making separation difficult (see, e.g. Caparon, et al (2010) Biotechnol. Bioeng. 105:239-249). The interaction of apolipoproteins with impurities increases the complexity of the purification process and can incur significant yield losses leading to a low throughput, high cost manufacturing process (see, e.g Hunter, et al (2009) Biotechnology Progress 25(2): 446-453).

Human apolipoprotein L1 (h-ApoL1) is a minor protein component of high density lipoprotein (HDL) particles and believed to play a role in lipid transport and metabolism (see, e.g., Duchateau, et al (1997) J. Biol. Chem. 272:25576-25582). Current studies have shown a link between variants of h-ApoL1 and the pathogenesis of non-diabetic chronic kidney disease (see, e.g., Genovese, et al. (2010) Science 329:841-845). African Americans carrying two ApoL1 risk alleles, termed G1 and G2, have a greatly increased risk for glomerular disease. The G1 variant has two missense mutations (S342G and I384M), while the G2 variant which has a 6 base pair (bp) in-frame deletion resulting in the deletion of 2 amino acids (N388del:Y389del). In the population carrying one or both risk alleles, focal segmental glomerulosclerosis (FSGS) occurs earlier and progresses to end-stage renal disease (ESRD) more rapidly (see, e.g, Kopp et al (2011) J. Am. Soc. Nephrol. 22:2129-2137).

Apolipoprotein L1 (ApoL1), is a genetically modified version of h-ApoL1 and its associated variants (e.g., EG, G1, G2). Bioactivity of ApoL1 purification preparations is assessed using a cell based Trypanosoma brucei since the colicin pore forming domain of ApoL1 will enhance influx of chloride ions into the cell membrane and cause cell death (see, e.g. Perez-Morga et al (2005) Science 309(5733): 469-472).

ApoL1 and h-APOL1 both possess multiple amphiphatic alpha helices, which leads to a reversible self-association phenomenon and formation of miscelle structures in solution similar to the behavior of other apolipoproteins previously described in the literature (see e.g. Zehender et al (2012) Biochemistry 51: 1269-1280 and Calabresi et al (1994) J. Biol. Chem. 269: 32168-32174). ApoL1 in its predominant form exists as approximately ≧15 protein molecules associated together. ApoL1 monomer consists of 372 amino acids with a molecular weight of approximately 41 kDa and a theoretical isoelectric point (pI) of about 5.49. Baboon ApoL1 and human ApoL2 also exist as multimeric protein molecules in solution with approximate molecular weights of 40 kDa and 37 kDa with a theoretical pI of about 6.52 and 6.31 respectively.

Chromatographic refolding of proteins by loading an unfolded protein and decreasing the denaturant concentration has been described in the literature for a variety of proteins including histidine-tagged proteins and recombinant proteins (see, e.g. Zhu, et al. (2005) Acta Biochim. et Biophys. Sinica. 37(4): 265-269, e.g. Cabanne, et al. (2005) J. Chromatogr. B. 818: 23-27). The majority of these proteins contain cysteine bonds, which need to be refolded to form the correct disulfide bonds within the protein structure. However, apolipoproteins consist of multiple amphiphatic alpha helices, which are typically refolded after purification.

Previous methods to purify apolipoproteins consisted of minimizing protein self-association using high urea concentrations to remove process impurities and often involved multiple steps (e.g. Hunter, et al (2008) J. Chromatogr. A. 1204:42-47 and Bankston, et al (2010) Biotech. J. 5: 1028-1039). Current methods to purify ApoL1 involve using an N-terminal histidine tag version of the protein in combination with immobilized metal ion affinity chromatography (IMAC) to enhance protein purity. Histidine tags attached to proteins enable an alternative purification protocol but can lead to immunogenicity. Since the ApoL1 protein does not significantly dissociate in high urea concentrations, different approaches were needed for removal of process related impurities. The current approach involves selection of excipients (i.e., detergent and denaturant combinations) and chromatography resins and membranes based on their ability to amplify the excipient interactions with ApoL1 for selected clearance of a process impurity based on the protein's folded state. The present invention fills this unmet need by providing a methodology to purify highly self-associating apolipoproteins.

### DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is the comparison of ApoL1 EIK and KIK isoforms.

FIGS. 2A-2C are the RP-HPLC profiles of EIK WT, G1 and G2, respectively, HIC feed and eluent.

FIG. 3 is the Circular Dichroism profile of APOL1 WT in various buffer matrices. Buffer conditions are represented as follows: 3M guanidine hydrochloride, open triangle (Δ), 8M urea, 2 w/v % SDS, open square (□), and HIC eluent, open circle (◯).

FIGS. 4A-4B are the RP-HPLC profiles of APOL2 HIC and ApoL2 Baboon feed and eluent.

FIG. 5 is the reduced SDS-Page gel of APOL1 wild type, APOL2 and APOL1 Baboon.

FIGS. 6A-6B are the circular dichroism profiles of ApoL1 Baboon and ApoL2.

FIGS. 7A-7B are the tryptophan fluorescence profiles of ApoL1 Baboon and ApoL2.

FIG. 8 is a schematic of the apolipoprotein chromatography purification process.

FIG. 9 is the bioactivity profiles for APOL1 wild type and APOL1 Baboon.

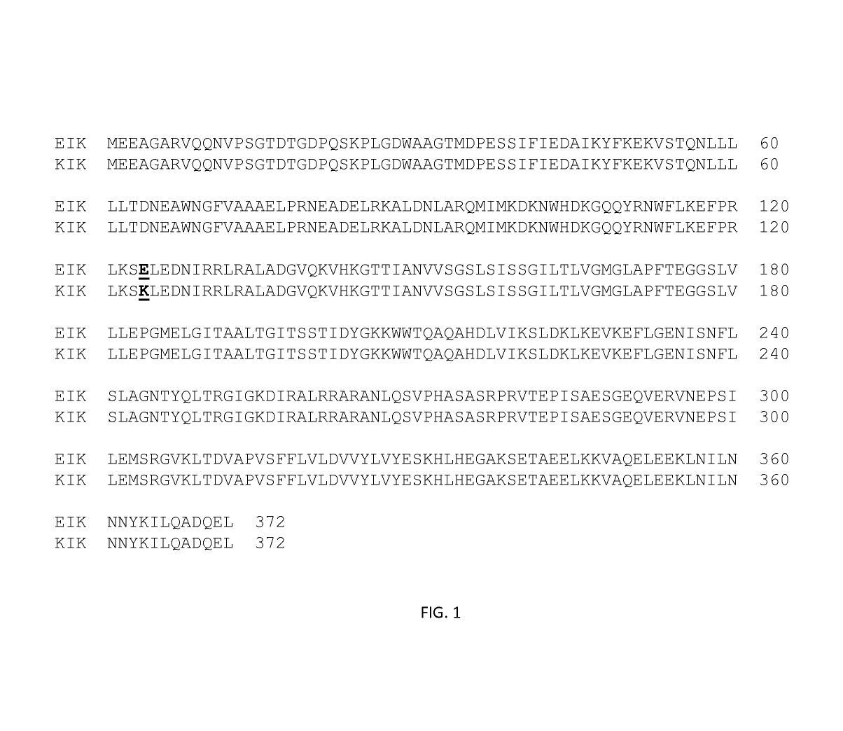

### SUMMARY OF THE INVENTION

The present invention is based, upon the discovery that lipoproteins can be refolded and purified using specialized chromatography steps. The present invention provides a method of refolding and purifying a lipoprotein expressed in a bacterial cell comprising solubilizing and unfolding inclusion bodies in a solubilization buffer; adjusting conductivity of the buffer containing the lipoprotein to at least 65-130 mS/cm; loading the buffer containing the lipoprotein on a hydrophobic interaction chromatography (HIC) column; washing the HIC column with a wash buffer; and eluting the lipoprotein with either a gradient elution buffer or a non-gradient elution buffer thereby yielding a HIC column eluent with a purity of at least 44%, as determine by reverse phase high performance liquid chromatography (RP-HPLC). In certain the bacterial cell is an E. coli cell; and the solubilization buffer comprises 2% SDS, 100 mM Tris, 100 mM NaCl, 200 mM Arginine, and 8M Urea at pH of 7 to 10. In a further embodiment, the lipoprotein is incubated in the solubilization buffer for one to twenty four hours at 15 to 30° C. In a further embodiment, dithiothreitol (1 mM) is added to the unfold buffer for apolipoproteins containing cysteine residues.

The present invention the also contemplates that the conductivity of the buffer containing the lipoprotein is adjusted to 65-130 mS/cm with 20 mM sodium phosphate and 1 to 2M ammonium sulfate. In further embodiments, the HIC column is loaded with up to 15 grams of apolipoprotein per liter of HIC resin; the wash buffer comprises 1 M ammonium sulfate at pH 7, and the gradient elution buffer comprises a gradient from about 20 mM sodium phosphate and 1 M ammonium sulfate at pH 7 (Buffer A), to about 20 mM sodium phosphate at pH 7 (Buffer B), and the lipoprotein is eluted over 20 column volumes. In other embodiments, Buffer A comprises 20 mM sodium phosphate and 0.2 to 0.3 M ammonium sulfate at pH 7 and Buffer B comprises 20 mM sodium phosphate at pH 7 with a concentration of 0 to 8M urea. In other embodiments, Buffer A and Buffer B both comprise of 0 to 8M urea.

The present invention further provides a method of purifying an apolipoprotein comprising: loading the HIC column eluent containing an apoliprotein in a flowthrough mode on a salt tolerant anion exchange chromatography membrane at feed conditions of pH 6.5 to 8.0 and conductivity of ≦5 to 30 mS/cm with a membrane loading of at least 50 milligrams of apolipoprotein per mL of membrane; washing the membrane with 20 mm HEPES and 200 mM NaCl at pH of 6.5-8.0; and collecting the flowthrough.

In yet further embodiments, the loading conditions are pH 6.5 to 7.5 and conductivity of ≦5 to 20 mS/cm; the anion exchange membrane is a salt tolerant anion exchange membrane; anion exchange membrane loading is about 200 milligrams of apolipoprotein per mL of membrane; and the apolipoprotein is human or baboon ApoL1 or human ApoL2.

The present invention also provides a method of removal of endotoxin for an apolipoprotein comprising: unfolding the apolipoprotein by addition of any combination of guanidine hydrochloride, urea, and ammonium sulfate to an apolipoprotein containing pool collected from an anion exchange membrane; loading the apolipoprotein in a flowthrough mode on a hydrophobic interaction chromatography (HIC) column; washing the HIC column with an appropriate wash buffer; and collecting the flowthrough. In other embodiments, apolipoprotein containing pool is loaded on the HIC column at conditions of: pH 6.0-7.5; b) conductivity of 130-170 mS/cm; 2 to 6M guanidine HCl; loading the apolipoprotein at 0.5 to 3 grams of apolipoprotein per liter of resin; 0 to 0.7M ammonium sulfate; and 0 to 2.5M urea.

In yet further embodiments, the hydrophobic interaction chromatography wash buffer is 20 mM sodium phosphate, 6M guanidine HCl, 0.75M ammonium sulfate pH 7; and the apolipoprotein is human or baboon ApoL1 or human ApoL2.

### DETAILED DESCRIPTION

As used herein, including the appended claims, the singular forms of words such as “a,” “an,” and “the,” include their corresponding plural references unless the context clearly dictates otherwise.

As used herein “ApoL1” refers to Apolipoprotein 1, and includes the wild type human isoforms EIK and KIK as well as the variants G1 and G2. ApoL1 can also refer to the genetically modified wild type or variant proteins, where either the N-terminal methionine or alanine are deleted. Non-human primate versions of ApoL1, modified or unmodified, are also included.

As used herein, the terms “about” or “approximately” used with a pH or pI (isoelectric point) value refers to a variance of 0.1 to 0.5 units. When used with a temperature value, “about” or “approximately” refers to a variance of 1 to 5 degrees. When used with other values, such as length, weight or concentration, “about” or “approximately” refers to a variance from 1 to 10%.

As used herein, a “mixture” comprises an apolipoprotein of interest (for which purification is desired) and one or more contaminant, i.e., impurities. A mixture that has been “partially purified” has already been subjected to a chromatography step, e.g., hydrophobic interaction chromatography, ion exchange chromatography, etc.

The term “chromatography” refers to any kind of technique which separates an analyte of interest (e.g., an apolipoprotein) from other molecules present in a mixture.

The term “chromatography resin” or “chromatography media” are used interchangeable herein and refer to any kind of solid phase which separates an analyte of interest from other molecules present in a mixture. Usually, the analyte of interest is separated from other molecules as a result of differences in rates at which the individual molecules of the mixture migrate through a stationary solid phase under the influence of a moving phase, or in bind and elute processes. Non-limiting examples include anion exchange resins, anion exchange membranes, and hydrophobic interaction resins. The volume of the resin, the length and diameter of the column to be used, as well as the dynamic capacity and flow rate depend on several parameters such as the volume of fluid to be treated, concentration of protein in the fluid to be subjected to the process of the invention, etc. Determination of these parameters for each step is well within the average skills of the person skilled in the art.

The term “host cell proteins” refers to proteins, other than the target protein, found in a lysate of the host cell. The amount of host cell protein in a protein mixture is expressed in parts per million relative to the amount of the protein of interest in the mixture.

The terms “target protein” or “protein of interest” as used interchangeably herein, refer to a protein or polypeptide, including but not limited to, a recombinant apolipoprotein that is to be purified by a method of the invention, from a mixture of proteins, and optionally, other materials such as host cell proteins, DNA, endotoxin, and the like.

As used herein, the terms “anion exchange” and “anion exchange membrane chromatography” are used to refer to a chromatographic process in which a solute or analyte of interest in a mixture interacts with a charged compound linked (such as by covalent attachment) to a membrane such that the solute or analyte of interest interacts non-specifically with the charged compound more or less than solute impurities or contaminants in the mixture. The contaminating solutes in the mixture elute from a membrane of the ion exchange material faster or slower than the solute of interest or are bound to or excluded from the resin relative to the solute of interest.

As used herein, the term “hydrophobic interaction chromatography” is used to refer to a chromatographic process wherein at high salt concentrations, the nonpolar groups on the surface of a protein interacts with a hydrophobic compound linked to a solid phase chromatographic media. An elution buffer with a lower salt concentration is used to separate proteins and contaminants based on their hydrophobicity with higher hydrophobic proteins eluting later in the chromatography gradient.

By “binding” a molecule to a chromatography resin is meant exposing the molecule to the chromatography resin under appropriate conditions (pH and conductivity) such that the molecule is reversibly immobilized in or on the chromatography resin by virtue of ligand-protein interactions. Non-limiting examples include hydrophobic interactions between the molecule and the hydrophobic ligands on the resin.

The terms “flowthrough” and “flowthrough mode” as used interchangeably herein, to refer to a product separation technique in which at least one product (apolipoprotein) contained in a sample along with one or more contaminants is intended to flowthrough a chromatographic resin or media, while at least one potential contaminant or impurity binds to the chromatographic resin or media. The “flowthrough mode” is generally an isocratic operation (i.e., a chromatography process during which the composition of the mobile phase is not changed).

As used herein, the term “elute” refers to a process which removes a protein of interest from a chromatography resin by altering the solution conditions such that the buffer competes with the molecule of interest for ligand sites on the chromatography resin.

The term “bind and elute mode” refer to a product separation technique in which at least one product contained in a sample binds to a chromatographic resin or media and is subsequently eluted.

The terms “contaminant” and “impurity” as used interchangeably herein, refer to any foreign or objectionable molecule, including a biological macromolecule such as DNA, one or more host cell proteins, endotoxins, or lipids in a sample containing the apolipoprotein of interest that is being separated from one or more of the foreign or objectionable molecules using a process of the present invention. Additionally, such a contaminant may include any reagent which is used in a step which may occur prior to the purification process.

The “isoelectric point” or “pI” of a protein refers to the pH at which the protein has a net overall charge equal to zero, i.e. the pH at which the protein has an equal number of positive and negative charges.

As used herein, the term “buffer” refers to a solution that resists changes in pH by the action of its acid-base conjugate components.

As used herein, the term “wash buffer” refers to a buffer used to wash or equilibrate the chromatography resin prior to eluting the protein of interest.

An “elution buffer” is used to elute the target protein from the solid phase. The conductivity and/or pH of the elution buffer is/are usually such that the target protein is eluted from the chromatography resin. The term “isocratic elution” is used to refer to an elution condition in which the composition of the mobile phase is unchanged during the entire elution process. The term “gradient elution” is used to refer to an elution condition in which the composition of the mobile phase changes during the entire elution process.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

All references cited herein are incorporated by reference to the same extent as if each individual publication, patent application, or patent, was specifically and individually indicated to be incorporated by reference.

### General

The present invention provides methods of refolding and purifying lipoproteins on a chromatography column. In particular methods of refolding ApoL1 polypeptides using a hydrophobic interaction column (HIC).

ApoL1 has a molecular weight of approximately 41 kDa and a theoretical isoelectric point of 5.49. Apo-L1 contains three main domains: Colicin pore-forming domain, membrane-addressing domain, and SRA-interacting domain. The sequences of human ApoL1 wild type isoforms EIK and KIK, as well as variants G1 and G2 (“Met”) are SEQ ID NOs.: 1, 3, 5, and 7. Additionally, engineered forms of ApoL1 isoforms and variants (“Ala”) are also provided in SEQ ID NO 2, 4, 6, and 8. Baboon ApoL1 (Met and Ala) are SEQ ID NOs: 9-10. A related apolipoprotien, ApoL2 is SEQ ID NO: 11.

The colicin pore-forming domain significantly enhances the cytotoxicity of Apo-L1 since colicins have the ability to rupture cytoplasmic membranes. However, this enhanced cytotoxicity makes production of Apo-L1 in E. coli extremely challenging due to the disruption of membrane integrity from Apo-L1.

Several methods, including dilution, dialysis, diafiltration, gel filtration, and immobilization onto a solid support, may be employed to remove or reduce excess denaturing and reducing agents, allowing proteins to renature. Dilution of the denatured solution directly into renaturation buffer is the easiest process but additional purification is needed after refolding. In dialysis, the denatured protein solution is dialyzed against renaturation buffer but lacks reproducibility and scalability since the dialysis rate is not controlled. Diafiltration may be an alternative approach since the rate of denaturant removal is not diffusion limited. However, accumulation of denatured protein on the membrane may limit its application.

Gel filtration chromatography has been successfully used to refold secretory leukocyte protease inhibitor, carbonic anhydrase and lysozyme but may also cause protein aggregation dependent upon buffer exchange conditions (Buchner (1991) Biotechnol. 9:157-162; Hamaker, et al. (1996) Biotechno. Prog. 12:184-189; and Bates et al. (1997) J. Chromatog. A 766:109-119). Protein aggregation in a chromatographic column can be prevented by binding the protein to a stationary phase followed by elution (Negro, et al. (1997) Protein Engg. 10:593-599; Kim, et al. (1997) Protein Engg. 10:455-462; and Qiu, et al. (1997) Protein Engg. 10(suppl):33). Another key practical advantage with on-column refolding is that it allows for some degree of purification of the desired product in addition to buffer exchange.

Apo-L1 is produced intracellular in E. Coli as inclusion bodies. The first purification step involves inclusion body solubilization and alpha helix partial unfolding. The apolipoprotein is partially unfolded using a buffer matrix containing 100 mM Tris, 100 mM sodium chloride, 200 mM arginine hydrochloride, 8M urea, and 2 w/v % sodium dodecyl sulfate (SDS) at about a pH range of 7 to 10 for about one to twenty four hours at about a temperature between 15 to 30° C. For apolipoproteins containing cysteine residues, dithiothreitol (1 mM) is added to the unfold buffer and subsequent chromatography wash and elution buffers.

Due to the high hydrophobicity of the apolipoprotein, hydrophobic interaction chromatography (HIC) using Phenyl sepharose high performance resin is utilized to refold and purify Apo-L1. This on-column refold procedure for Apo-L1 varies in contrast to currently known methods for apolipoprotein purification and alpha helix unfold/refolding (Hunter, et al. (2008) J. Chromatog. A 1204:42-47; and Gross, et al. (2006) Biophys. J. 90:1362-70). While alternative hydrophobic resins that comprise of a different hydrophobic ligand may not behave similarly to the Phenyl Sepharose HP resin, optimal bind and elute conditions for the alternative hydrophobic resin can be determined using the methods disclosed herein. Commercially available examples of alternative hydrophobic resins include but are not limited to Butyl Sepharose HP, Capto Phenyl, Capto Butyl, Hexyl 650C, and Capto Octyl. The conductivity of the partially unfolded apolipoprotein was adjusted to between approximately 65 to 130 mS/cm using an ammonium sulfate stock solution and loaded onto the column at approximately a pH range of 6 to 8 and ≦11 grams of protein per liter of resin. After loading, the column is washed with about ≧3 column volumes (CVs) of 20 mM sodium phosphate, 1M ammonium sulfate pH 7. Elution is achieved using a linear gradient from 20 mM sodium phosphate, 1M ammonium sulfate pH 7 (Buffer A) to 20 mM sodium phosphate (Buffer B) over about 20 CVs. The elution gradient could vary from about 20 to 30% Buffer A to 100% Buffer B. Buffers A and/or B can also contain up to 8M urea. The column was regenerated with ≧3 CVs each of deionized water, 0.5M sodium hydroxide and 1M sodium chloride solution. The column was stored in 0.1M sodium hydroxide solution. Buffering compounds used in the hydrophobic interaction chromatography process include sodium phosphate, ammonium sulfate, and urea. Alternative buffering compounds may include but are not limited to HEPES, tris, citrate, acetate, sodium sulfate, sodium chloride, or some mixture of the foregoing or other buffers.

Anion exchange membrane chromatography is the subsequent purification step after hydrophobic interaction chromatography. The Sartobind salt tolerant anion exchange membrane is used to separate host cell protein from the apolipoprotein of interest. Using the methods disclosed herein, commercially available examples of alternative salt tolerant anion exchange membranes include but are not limited to Chromasorb. Anion exchange membrane chromatography was operated in a flowthrough mode using approximate feed conditions of pH 6.5 to 8.0 and 5 to 19 mS/cm at a membrane loading of ≦200 grams of protein per mL of membrane. Product collection started when the UV increased to 125 mAu/cm. After loading, the membrane is washed with 20 mM Hepes, 200 mM sodium chloride pH 7.4 until the UV dropped below 125 mAu/cm. The wash buffer may also contain 10 v % glycerol. The membrane was stripped using 1M sodium chloride solution. Buffering compounds used in the anion exchange membrane chromatography process include HEPES, sodium chloride, and glycerol. Alternative buffering compounds may include but are not limited to tris or acetate or some mixture of the foregoing or other buffers.

Hydrophobic interaction chromatography step operated in flowthrough mode is the final purification step. Phenyl sepharose HP or butyl sepharose HP is used to separate endotoxin from the apolipoprotein of interest. Using the methods disclosed herein, commercially available examples of alternative hydrophobic interaction chromatography resins include but are not limited to Capto Phenyl, Capto Butyl, Hexyl 650C, and Capto Octyl. The conductivity of the HIC feed is adjusted to approximately 160 mS/cm using any combination of urea, guanidine hydrochloride, and ammonium sulfate and loaded onto the column up to 3 grams of apolipoprotein per liter of resin. Product collection starts when the UV increased to 125 mAu/cm. After loading, the column was washed with 20 mM sodium phosphate, 6M guanidine hydrochloride, 0.75M ammonium sulfate pH 7 until the UV decreases below 125 mAu/cm. The column was regenerated with ≧3 CVs each of deionized water, 0.5M sodium hydroxide and 1M sodium chloride solution. Isopropanol (20 v %) is used as an alternative column regeneration solution. The column is stored in 0.1M sodium hydroxide solution. Buffering compounds used in the hydrophobic interaction chromatography process include sodium phosphate, ammonium sulfate, urea, and guanidine hydrochloride. Alternative buffering compounds may include but are not limited to HEPES, tris, citrate, acetate, sodium sulfate, sodium chloride, or some mixture of the foregoing or other buffers.

Removal of process impurities, such as endotoxin, host cell proteins, and protein fragments, is extremely complex and difficult to achieve using traditional chromatography protocols. To overcome purification challenges, the present invention involves regulating the self-association behavior of the molecule by identifying the appropriate excipients, such as detergent and denaturant combinations, which either causes a partial or full unfolding of the apolipoprotein. Chromatography resins and membranes were evaluated and selected based on their ability to amplify the excipient interactions with ApoL1 for selected clearance of a process impurity based on the protein's folded state. A 3-step purification scheme was developed to remove approximately 2 to 9 log₁₀ of endotoxin, host cell protein clearance up to 2 log₁₀, and resulted in a final purity of ≧44% by reverse phase HPLC. This purification process has been successfully applied to three human ApoL1 variants along with human ApoL2 and baboon ApoL1.

The broad scope of this invention is best understood with reference to the following examples, which are not intended to limit the inventions to the specific embodiments.

### EXAMPLES

### I. General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391).

### II. Materials

All apolipoproteins purified in these studies were produced in E. coli, and isolated as inclusion bodies. The ApoL1 proteins included: ApoL1 wild type isoforms (ie. EIK and KIK), the renal high risk variants (G1 and G2), ApoL2, and Baboon ApoL1. Phenyl Sepharose High Performance and Butyl Sepharose High Performance was from GE Healthcare (Piscataway, N.J., USA) and the Sartobind Salt Tolerant Anion Exchange (STIC) membrane was from Sartorius Stedim (Goettingen, Germany). All chemical reagents were purchased from Fisher Scientific (Pittsburgh, Pa., USA), Sigma Aldrich (St. Louis, Mo., USA), or Biorad (Hercules, Calif., USA).

### III. Chromatography

Chromatography separations were executed on GE Healthcare Avant systems using either Tricorn 5/200 or XK16, 26, or 50 columns from GE Healthcare. All studies used a linear velocity range of 30-250 cm/hr with a column loading of ≦11 grams of protein per liter of resin. Columns were packed to bed heights of 8-22 cm and packing efficiency was ensured by preset criteria of height equivalent to a theoretical plate (HETP) of ≧1000 plates per meter and peak asymmetry factor range of 0.8-1.8.

### IV. Analytical Methods

Analytical Reverse Phase High Performance Chromatography (RP-HPLC):

RP-HPLC was performed using a Poros R2/10 2.1×30 mm analytical column (Applied Biosystems) coupled with an in-line guard filter (ADV 0.5 μm Direct Connect In-Line Column Filter). Buffer A consisted of 0.2 v/v % TFA in water and buffer B consisted of 0.2 v/v % TFA in 90 v/v % acetonitrile. After sample injection onto the column, a gradient of increasing acetonitrile was used to elute the proteins based on hydrophobicity, with more hydrophobic species eluting later in the gradient. The RP-HPLC method consisted of a flowrate of 1 ml/min at 70° C. with a 24 minute run time and a 2 minute post run time. Protein concentration was measured at a UV signal of 280 nm wavelength.

Protein Quantification:

Total protein concentrations were determined via absorbance of 280 nm using a NanoDrop model 2000c from Thermo Fisher Scientific (Waltham, Mass., USA). The theoretical apolipoprotein extinction coefficients of 1.056 for ApoL1 variants, 0.627 for Baboon ApoL1, and 0.646 for ApoL2 were calculated from the amino acid sequence and used to determine protein concentration.

Circular Dichroism (CD):

Circular dichroism was performed on a spectrophotometer from Aviv Biomedical (420C). ApoL1 wild type was diluted 1:10 into different buffers. The ellipticity was monitored with a data pitch of 0.5 nm and averaging 2 s at each wavelength in the interval of 250 nm-198 nm (if possible) for both the buffer and the protein solutions. Three scans are averaged to obtain the final spectrum. Cell cuvettes with 1 mm pathlength are used with a bandwidth was set to 1 nm. For data analysis, the buffer scan is subtracted from the protein scans. The scans are normalized to molecular rest weight by using the equation:

$${Θ}_{MRW} = \frac{Θ100 MW}{c {dN}_{A}}$$

where Θ=measured ellipticity (°)
c=protein concentration (mg/ml),
d=path length (cm)
MW=protein molecular weight (g/mol)
and N_{A}=number of amino acids

Endotoxin Quantification:

Reduction of endotoxin for each process step is determined using either a portable Endosafe PTS reader (Charles River, Wilmington, Mass.) with disposable endotoxin cartridges or using an in-house kinetic assay that measures an ultraviolet wavelength of 405 nm over a period of two hours and is calibrated with a standard curve to determine endotoxin concentration.

Gel Electrophoresis (SDS-Page):

Apolipoprotein samples (50 microliters each) was diluted 1:1 with DI water (50 microliters) in separate 2 mL tubes. Tris Glycine SDS sample buffer 2λ (200 microliters) was diluted 1:1 with DI water (200 microliters) in a separate 2 mL tube. The diluted Tris glycine SDS sample buffer (19 microliters) is added to separate 2 mL tubes. The following volumes of apolipoprotein samples are added to separate tubes to equal 0.5 microgram and 1.0 microgram gel loading respectively: EIK WT (0.8 and 1.4 microliters), ApoL2 (1.8 and 3.4 microliters), Baboon ApoL1 (1.2 and 2.5 microliters). Mercaptoethanol (1 microliter) is added to each sample containing SDS sample buffer. All samples are vortexed and heated to 95° C. for 3 minutes. Samples are loaded into a 1.0 mm×10 well Novex 4-20% Tris-glycine gel containing 1× Tris glycine SDS running buffer. A BioRad Precision Plus protein dual color standard (7 microliters) is added to the first and last wells in the gel. Gel electrophoresis is conducted at a current of 200 volts for 65 minutes. The gel is washed with deionized water three times, stained with simply blue safe stain for 60 minutes, and destained with deionized water.

Host Cell Protein Quantification:

Host cell protein levels in the product pools were determined using a commercial Cygnus kit.

Bioactivity Assay:

A trypanosome cell based assay was used to quantify apolipoprotein bioactivity with and without ammonium chloride inhibition.

### V. Results

Removal of Process Impurities from an Apolipoprotein Inclusion Body Preparation by Partial Unfolding and Hydrophobic Interaction Chromatography on-Column Refolding

IC experiments show resolution of product related fragments and impurities resulting in an apolipoprotein purity of at least 67 A % as detected by RPHPLC (Table 1).

TABLE 1
HIC Performance for APOL1 Variant Purifications

| | Approximate Purity by | |
|---|---|---|
| Apolipoprotein Variant | RP-HPLC (A %) | Yield (%) |
| EIK WT, Batch #1 | 74 | 73 |
| EIK WT, Batch #2 | 87 | 78 |
| KIK WT | 93 | 81 |
| G1 | 93 | 95 |
| G2 | 96 | 87 |

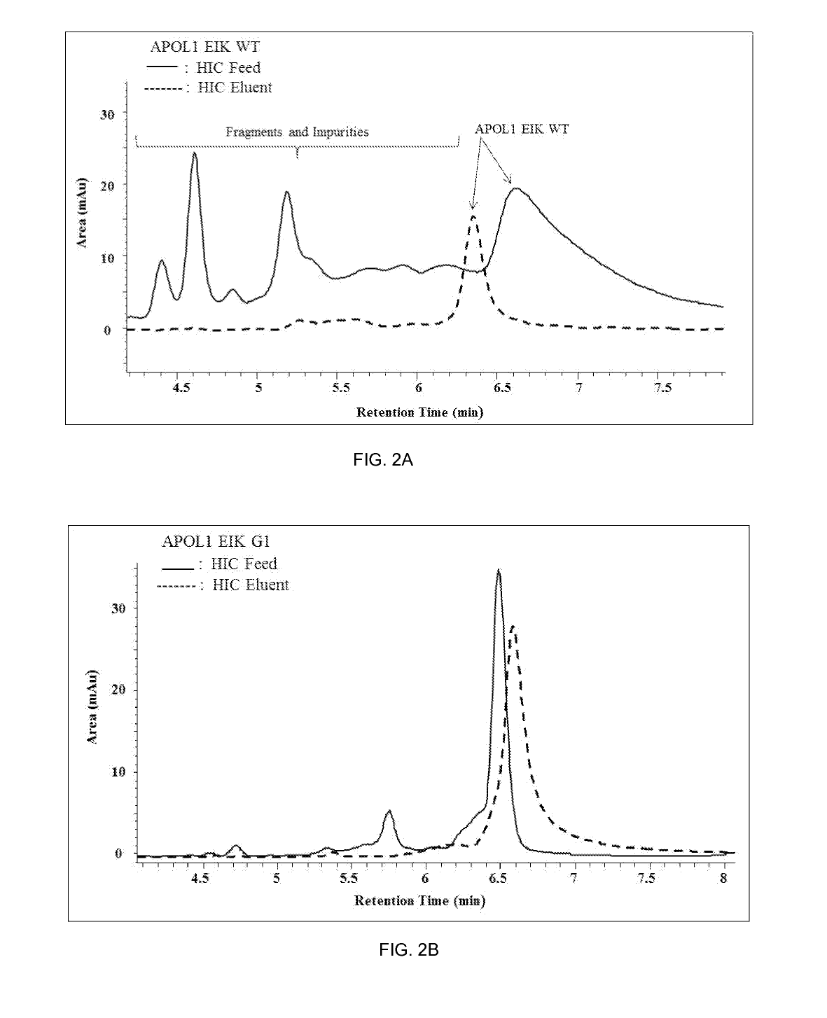

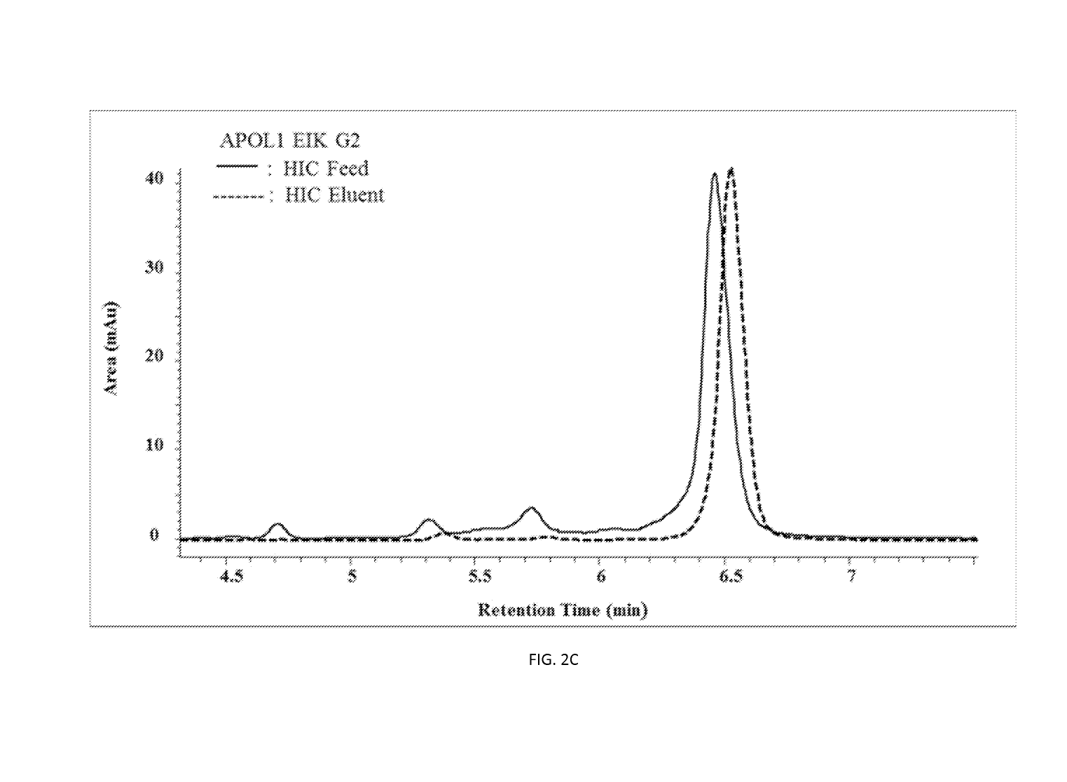

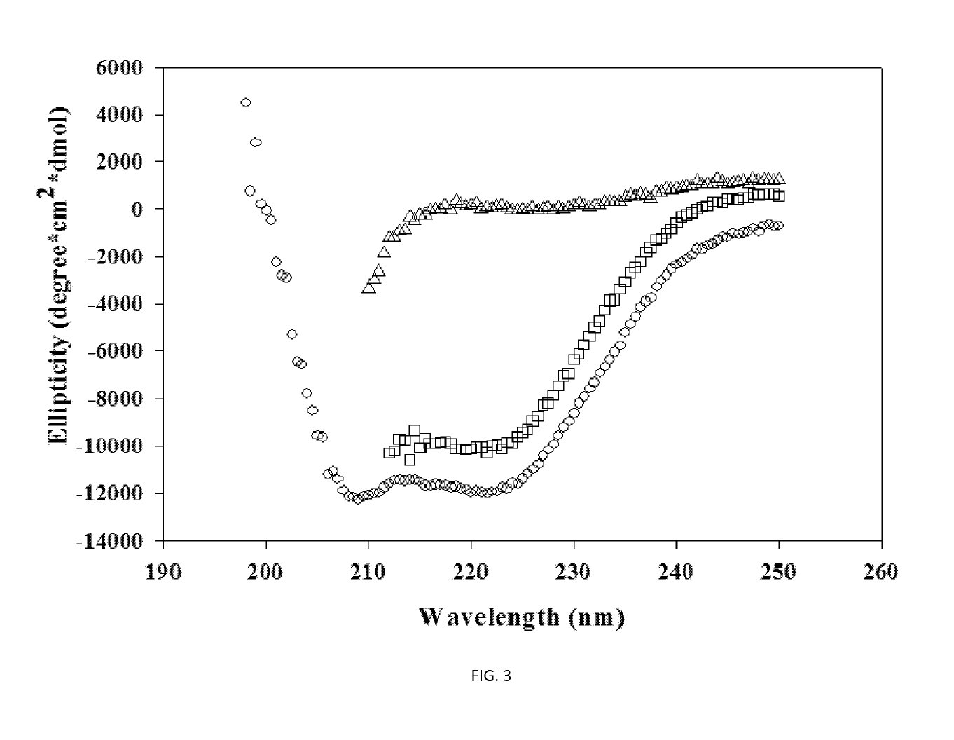

The majority of product fragments (retention time of 4.5-5.5 minutes) were separated from the APO-L1 eluent (approximate retention time of 6.4 minutes) as shown by the RP-HPLC chromatographs (FIGS. 2A-2C). The circular dichroism profile of the HIC eluent shows two inflection points in the spectral region of 190-250 nm, which is characteristic of a properly refolded, alpha helical protein (FIG. 3). The excipients in the unfold buffer allow the protein to reach a partially unfolded intermediate state as indicated by the rise of the circular dichroism profile (FIG. 3). Proper refolding of the apolipoprotein across the column in combination with a decreasing elution buffer concentration allows for removal of product fragments that are similar in molecular weight to ApoL1.

Hydrophobic interaction chromatography showed the ability to resolve protein fragments to enhance apolipoprotein purity while allowing the protein to refold into its native alpha helical structure. This on-column refolding protocol was successfully utilized for the purification of ApoL1 and its related variants (i.e. EIK, KIK, G1, G2).

On-Column Refolding and Purification of Apolipoproteins with Different Primary Amino Acid Sequences

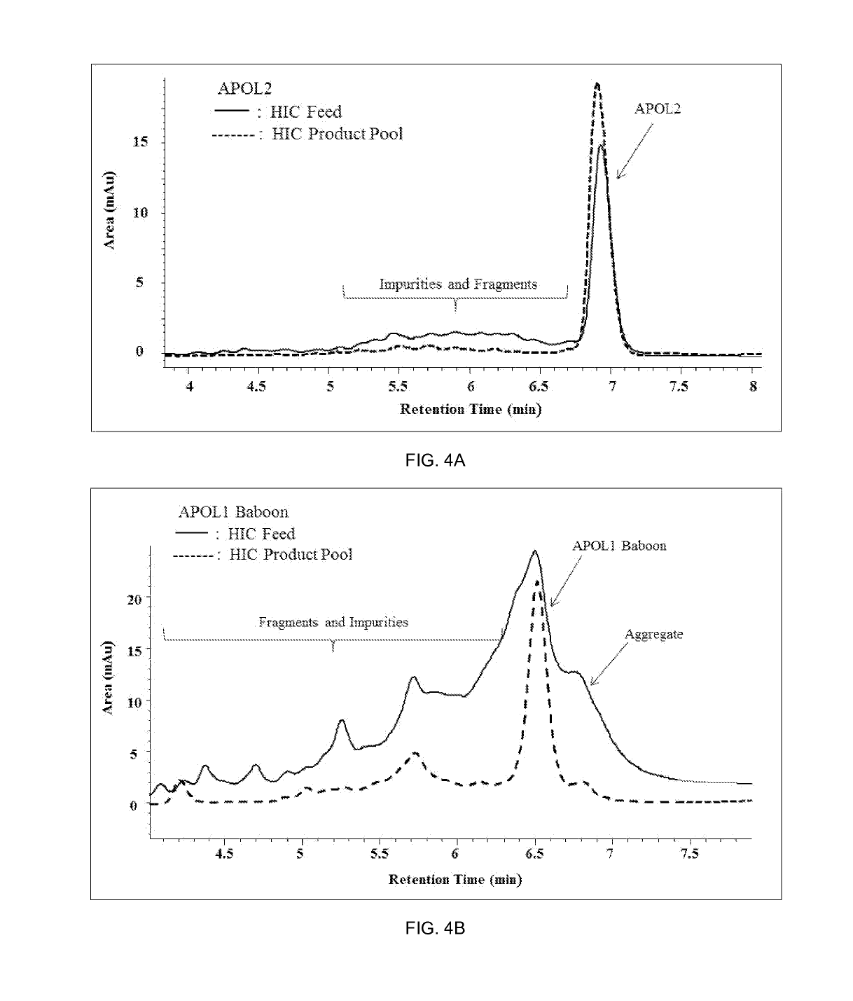

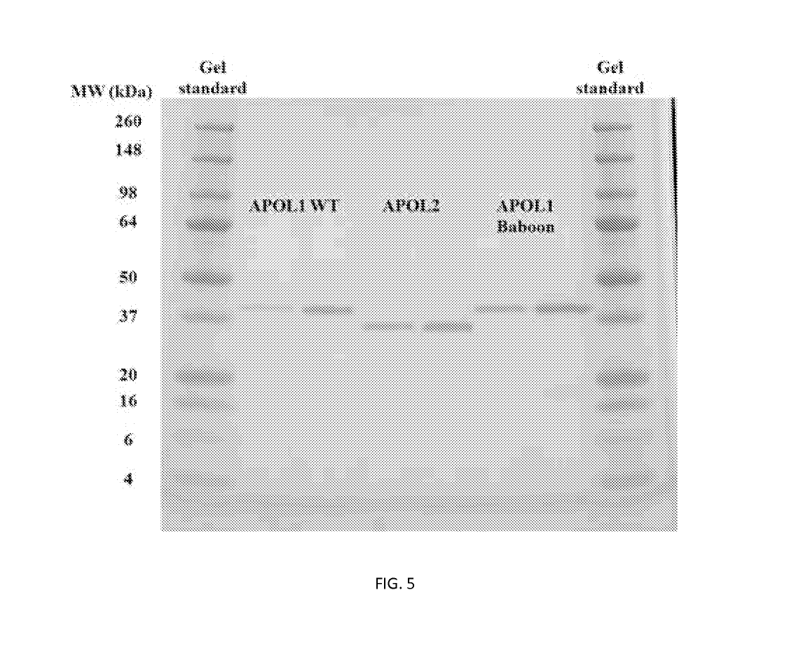

The majority of product related fragments were removed from the apolipoprotein of interest with RPHPLC purities ranging from 44-71% for Baboon ApoL1 dependent on feedstock and 80% for ApoL2 (Table 2, FIGS. 4A-4B).

TABLE 2
HIC Performance for Apolipoprotein Purifications

| | Approximate Purity by | |
|---|---|---|
| Apolipoproteins | RP-HPLC (A %) | Yield (%) |
| Baboon ApoL1, Batch #1 | 44 | 66 |
| Baboon ApoL1, Batch #2 | 71 | 51 |
| ApoL2 | 80 | 66 |

The on-column HIC refolding step demonstrated removal of the product related fragments and also had the ability to removal additional impurities such as endotoxin and host cell protein for apolipoproteins with different primary amino acid sequences. Therefore, this on-column refolding step can be applied to purify different types of apolipoproteins.

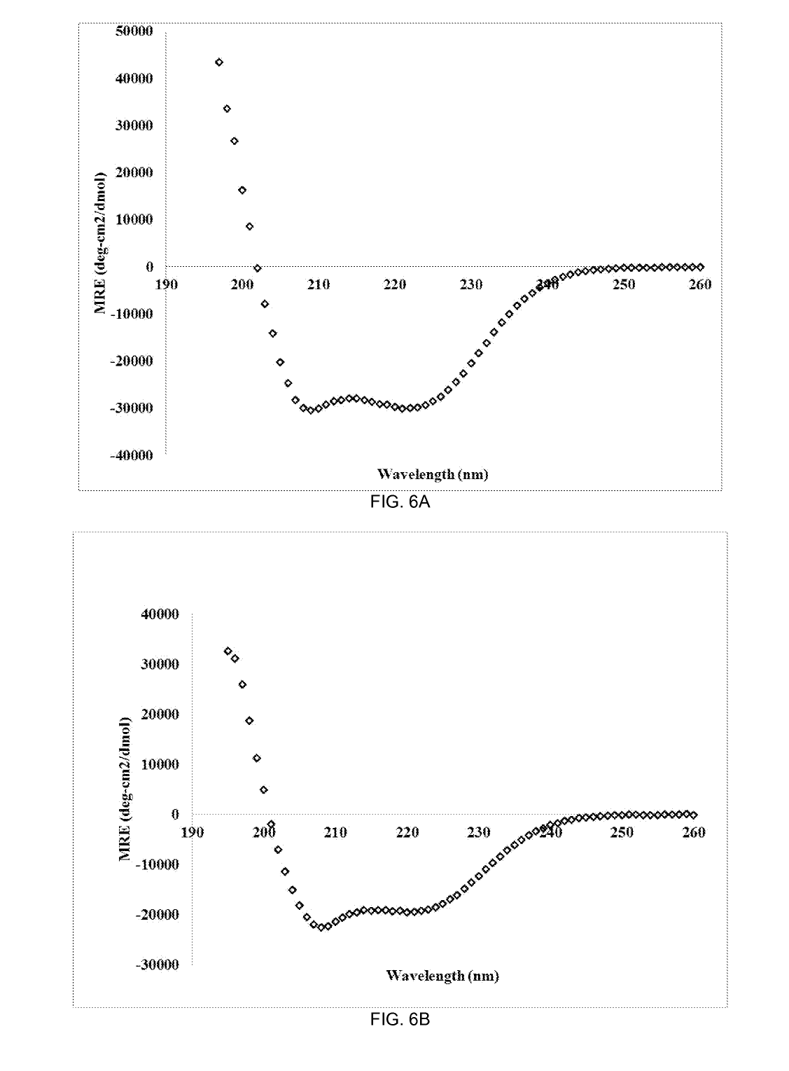

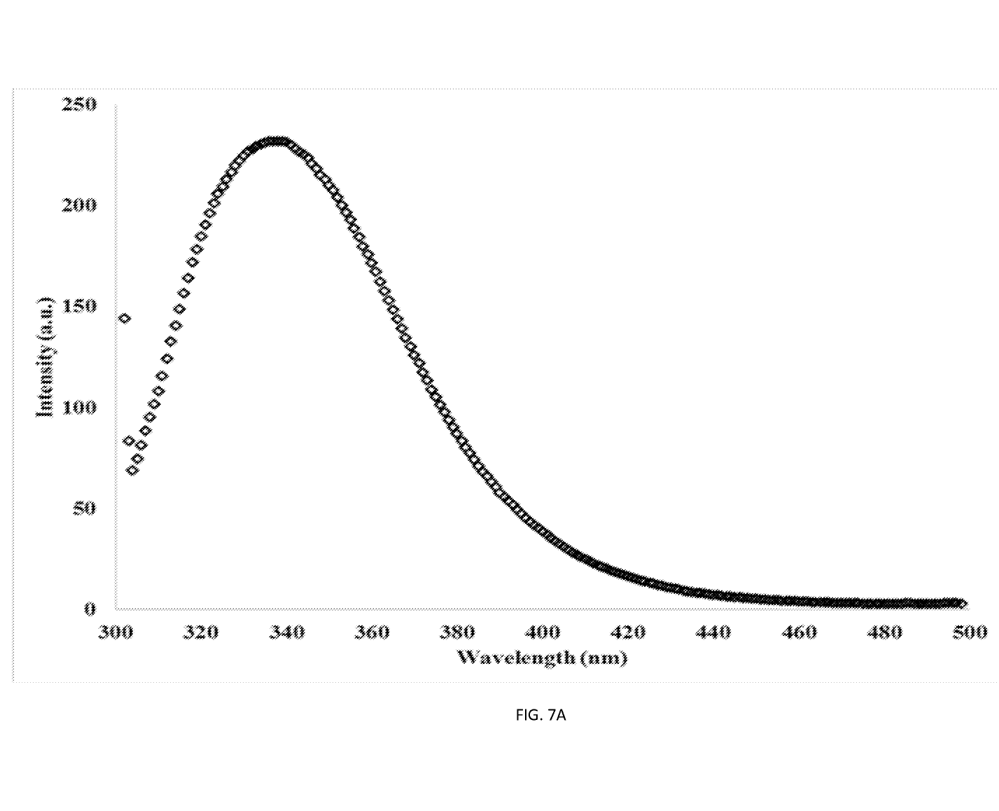

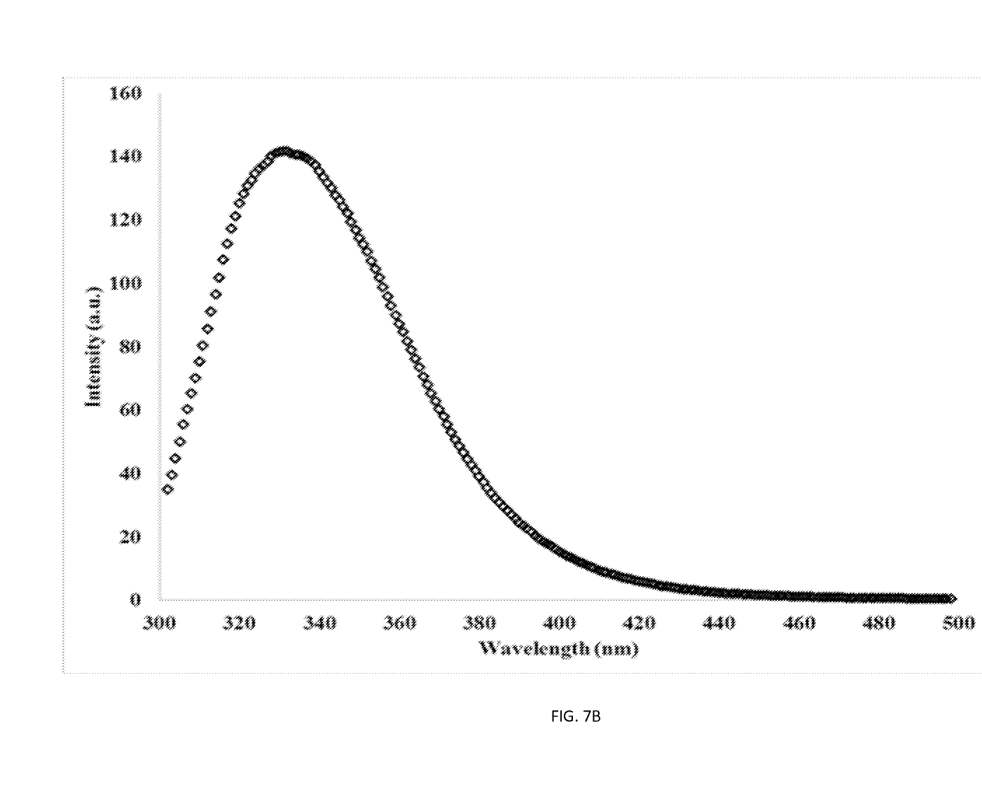

Additional impurities, such as host cell protein (HCP) and endotoxin were also cleared across the HIC column up to 1 and 2 log₁₀ respectively for Baboon ApoL1. In addition, a reduced SDS-gel shows that Baboon ApoL1 Batch #2 and ApoL2 demonstrate similar purity to ApoL1 wild type (FIG. 5). Circular dichroism and intrinsic tryptophan fluorescence spectroscopy profiles of the HIC product for both apolipoproteins indicate secondary and tertiary structural features typical of native alpha helical proteins (FIGS. 6A-6B, 7A-7B).

Endotoxin Removal Using a Combination of Complete Apolipoprotein Unfolding and Hydrophobic Chromatography in a Flowthrough Mode

Endotoxin removal for all apolipoproteins ranged from 1 to 3 log₁₀ (Table 3). The clearance of endotoxin is enhanced by completely unfolding the apolipoprotein using guanidine hydrochloride to allow for dissociation of endotoxin from the protein (FIG. 3). Feed conditions on hydrophobic interaction chromatography enabled the apolipoprotein to flowthrough the HIC column while selectively binding the endotoxin.

TABLE 3
Endotoxin Removal and HIC Flowthrough Performance Summary

| Apolipoprotein | Endotoxin Removal (log₁₀) | Yield (%) |
|---|---|---|
| EIK WT | 2-3 | ≧85 |
| KIK WT | 3 | |
| G1 | 3 | |
| G2 | 3 | |
| ApoL1 Baboon | 1 | |

The flowthrough HIC purification step resulted in 1-3 log₁₀ endotoxin removal for various types of apolipoproteins, which indicates this protocol can purify apolipoproteins composed of different primary sequences.

### Determination of Apolipoprotein Bioactivity

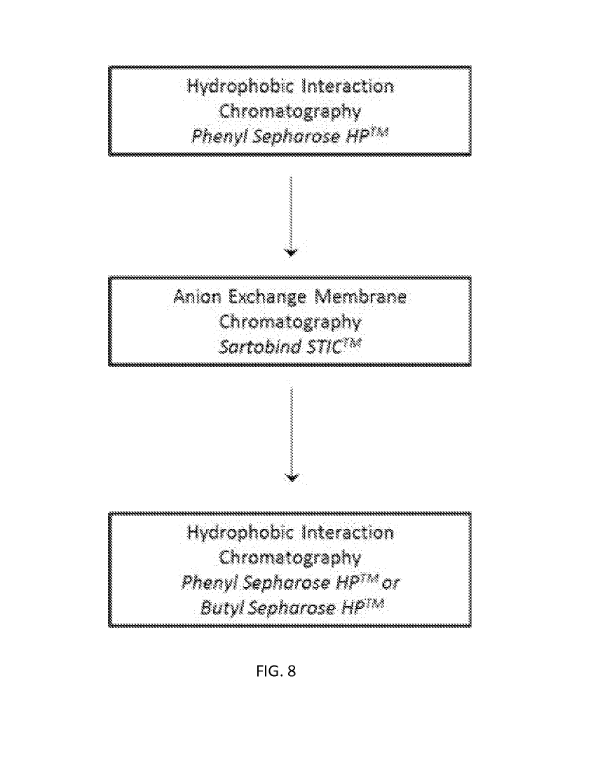

ApoL1 WT and Baboon ApoL1 were purified through the purification process shown in FIG. 8 with results summarized in Tables 4 and 5.

TABLE 4
Purification Performance Summary for ApoL1 WT

| | Endotoxin Reduction | HCP Reduction | Purity |
|---|---|---|---|
| Process Step | (log₁₀) | (log₁₀) | (A %) |
| HIC Bind and Elute | 3 to 6 | 2 | ≧74 |
| Anion Exchange | 1 to 2 | | |
| Membrane | | | |
| Chromatography | | | |
| HIC Flowthrough | 2 to 3 | | |

TABLE 5
Purification Performance Summary for ApoL1 Baboon

| | Endotoxin | HCP Reduction | Purity |
|---|---|---|---|
| Process Step | Reduction (log₁₀) | (log₁₀) | (A %) |
| HIC Bind and Elute | 2 | 1 | ≧44 |
| Anion Exchange Membrane | 1 | | |
| Chromatography | | | |
| HIC Flowthrough | 1 | | |

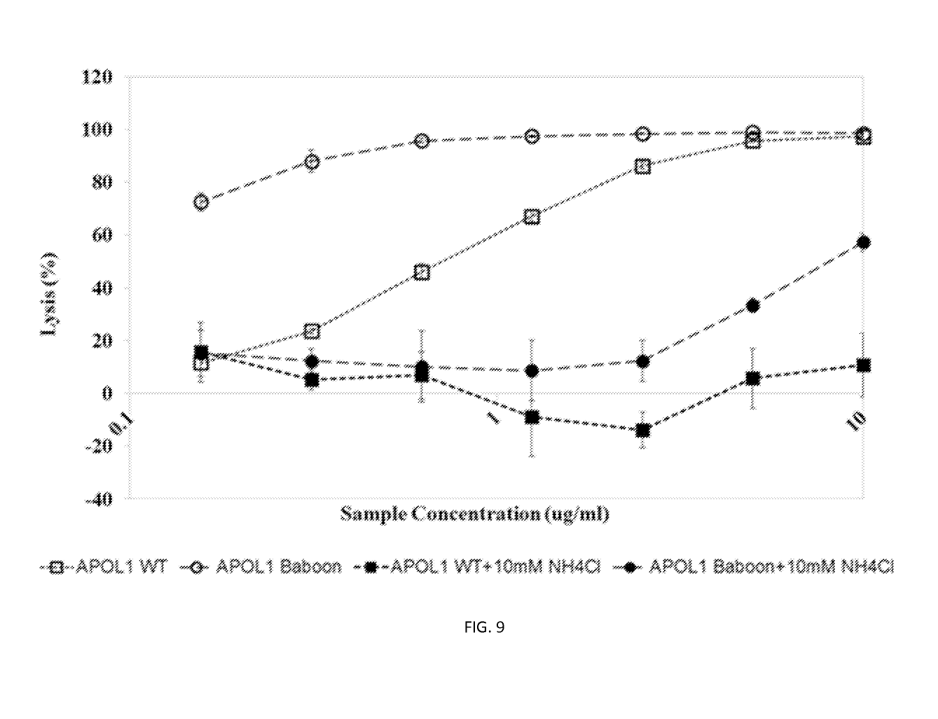

Both apolipoproteins were formulated and tested for bioactivity using a trypanosome cell based assay over a 24 hour time period. The bioactivity of APOL1 wild type (WT) and APOL1 Baboon was 5 μg/mL and 0.5 μg/mL respectively (FIG. 9). The addition of ammonium chloride inhibited trypanosome lysis to a greater extent for APOL1 WT at ≦10 μg/mL than APOL1 Baboon at ≦2.5 μg/mL.

ApoL1 wild type and Baboon ApoL1 show trypanosome lysis, which correlates to a level of bioactivity of 5 μg/mL and 0.5 μg/mL respectively. The purification process detailed in this patent can purify apolipoproteins composed of different primary sequences and also maintain protein bioactivity.

What is claimed is:

1. A method of refolding and purifying a lipoprotein expressed in a bacterial cell comprising:
a) solubilizing and unfolding inclusion bodies in a solubilization buffer;
b) adjusting conductivity of the buffer containing the lipoprotein to at least 65-130 mS/cm;
c) binding the lipoprotein on a hydrophobic interaction chromatography (HIC) column;
d) washing the HIC column with a wash buffer; and
e) eluting the lipoprotein with either a gradient elution buffer or a non-gradient elution buffer thereby yielding a HIC column eluent with a purity of at least 44%, as determined by reverse phase high performance liquid chromatography (RP-HPLC).

2. The method of claim 1, wherein the bacterial cell is an E. coli cell.

3. The method of claim 1 wherein the solubilization buffer comprises 2% SDS, 100 mM Tris, 100 mM NaCl, 200 mM Arginine, and 8M Urea at pH of 7 to 10.

4. The method of claim 3, wherein the lipoprotein is incubated in the solubilization buffer for at one to twenty-four hours at 15-30° C.

5. The method of claim 1, wherein the conductivity of the buffer containing the lipoprotein is adjusted to 65-100 mS/cm with 20 mM sodium phosphate and 1-2M ammonium sulfate.

6. The method of claim 1 wherein the HIC column is loaded with up to 15 grams of lipoprotein per liter of HIC resin.

7. The method of claim 1, wherein the wash buffer comprises 1 M ammonium sulfate at pH 7.

8. The method of claim 1 wherein the gradient elution buffer comprises a gradient from about 20 mM sodium phosphate and 1 M ammonium sulfate at pH 7 (Buffer A), to about 20 mM sodium phosphate at pH 7 (Buffer B), and the lipoprotein is eluted over 20 column volumes.

9. The method of claim 8, wherein Buffer A comprises 20 mM sodium phosphate and 0.2 to 0.3 M ammonium sulfate at pH 7 with a concentration of 0 to 8M urea.

10. The method of claim 8 wherein the gradient elution Buffer B comprises 20 mM sodium phosphate at pH 7 with a concentration of 0 to 8M urea.

11. A method of purifying an apolipoprotein comprising:
a) loading the HIC column eluent of claim 1 in a flowthrough mode onto an anion exchange membrane at feed conditions of pH 6.5-8.0 and conductivity of ≦5 to 30 mS/cm with a membrane loading of at least 50 mg protein per mL of membrane;
b) washing the membrane with 20 mm HEPES and 200 mM NaCl at pH of 6.5-8.0; and
c) collecting the flowthrough.

12. The method of claim 11 wherein the loading conditions are pH 6.5-7.5 and conductivity of ≦5-20 mS/cm.

13. The method of claim 11 wherein the anion exchange membrane is a salt tolerant anion exchange membrane.

14. The method of claim 11 wherein the anion exchange membrane loading is about 200 mg of protein per mL of membrane.

15. The method of claim 11, wherein the apolipoprotein is ApoL1 or ApoL2.

16. A method of removal of endotoxin for an apolipoprotein comprising:
a) unfolding the apolipoprotein by addition of guanidine hydrochloride to an apolipoprotein containing pool collected from an anion exchange membrane;
b) adding urea and ammonium sulfate to the pool;
c) loading the apolipoprotein in a flowthrough mode on a hydrophobic interaction chromatography (HIC) column;
d) washing the HIC column with an appropriate wash buffer; and
d) collecting the flowthrough.

17. The method of claim 16 wherein the apoliprotein containing pool is flowed through the HIC column at conditions of:
a) pH 6.0-7.5;
b) conductivity of 130-170 mS/cm;
c) 2 to 6M guanidine HCl;
d) loading the apoliprotein at 0.5-3 grams of apolipoprotein per liter of resin;
e) 0 to 0.7M ammonium sulfate; and
f) 0 to 2.5M urea.

18. The method of claim 17 wherein the hydrophobic interaction chromatography wash buffer is 20 mM sodium phosphate, 6M guanidine HCl, 0.75M ammonium sulfate pH 7.

19. The method of claim 17, wherein the apolipoprotein is ApoL1 or ApoL2.
